(19) United States
(12) Patent Application Publication
(10) Pub. No.: US 2008/0139664 A1
(43) Pub. Date: Jun. 12, 2008

(54) ANTI-GLYCATION AGENTS FOR PREVENTING AGE-, DIABETES-, AND SMOKING-RELATED COMPLICATIONS

(75) Inventors: Faustinus YEBOAH, Longueuil (CA); Yasuo KONISHI, Kirkland (CA); Sung Ju CHO, Montreal (CA); Jittiwud LERTVORACHON, Montreal (CA); Taira KIYOTA, St. Laurent (CA); Tomasz Popek, Pointe-Claire (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, omitted

(73) Assignee: NATIONAL RESEARCH COUNCIL OF CANADA, Ottawa (CA)

(21) Appl. No.: 12/029,209

(22) Filed: Feb. 11, 2008

Related U.S. Application Data
(62) Division of application No. 10/492,553, filed on Oct. 8, 2004.
(60) Provisional application No. 60/328,808, filed on Oct. 15, 2001.

(51) Int. Cl. A61K 31/135; A61P 3/00

(52) U.S. Cl. 514/646

(57) ABSTRACT

The invention provides new inhibitors of protein glycation, identified from compound libraries by a high throughput screening assay. The anti-glycation agents so identified are characterized by a variety of chemical structures and are useful for the prevention or treatment of age-, diabetes-, and smoking-related complications, including neuropathy, nephropathy, ocular pathologies, or the loss of mechanical properties of collagenous tissues. Among compounds identified as having the anti-glycation activity, of special interest are epinephrine and its analogs, in particular D-epinephrine and its analogs, which are particularly useful for the prevention or treatment of age-, diabetes-, and smoking-related ocular pathologies.

### CROSS REFERENCE TO RELATED APPLICATIONS

This application is a divisional application of U.S. Ser. No. 10/492,553, the disclosure of which is incorporated by reference in its entirety and is a US National Entry of PCT/CA02/01552 filed 15 Oct. 2002 and claims the benefit of U.S. Provisional Application No. 60/328,808 filed 15 Oct. 2001, the disclosure of which is incorporated by reference in its entirety.

### FIELD OF THE INVENTION

The invention relates to inhibitors of glycation of proteins, lipids, and nucleic acids and use thereof for prevention and treatment of age-, diabetes-, and smoking-related complications, in particular ocular pathologies.

### BACKGROUND OF THE INVENTION

In the past two decades, there has been a growing body of evidence implicating the glycation of body proteins in the development of micro- and macro-vascular complications underlying such disease states as nephropathy, neuropathy, and atherosclerotic disorders associated with diabetes and normal ageing (for a recent review, see Singh, R. et al., Diabetologia 44, 129-146 (2001)). The major complications include functional impairment of the cardiovascular system, kidney dysfunction, vision impairment, and the loss of mechanical properties of collagenous tissues, such as cartilage.

Glycation is a non-enzymatic or chemical process initiated by the interaction between reducing sugars, such as glucose, and primary amino groups of proteins, lipids and nucleic acids. In the initial reaction between primary amino groups of proteins (especially the ε-amino group of lysine residues) and the carbonyl group of reducing sugars a Schiff base is formed. The reaction then proceeds through a series of reversible rearrangements to form a metastable intermediates referred to as Amadori products (AP). With time, AP undergo oxidative degradation that leads to the formation of inter- and intra-protein cross-links and low molecular weight fragmentation products, collectively referred to as advanced glycation endproducts (AGEs). Some of the low molecular weight AGEs contain α-dicarbonyl group and are highly reactive oxidizing agents. AGEs readily interact with and modify proteins, lipids and nucleic acids, and increase the oxidative stress of biological systems.

Although all tissue and serum proteins are susceptible to non-enzymatic glyco-modification, the deleterious effects of glycation are more pronounced with long-lived proteins, such as collagen and lens crystallins. Furthermore, a receptor for AGEs (RAGE) has been identified. Upon binding of AGEs, the receptor up-regulates its expression and triggers an ascending spiral of cellular perturbations due to sustained RAGE-mediated cellular activation. Though further studies are required to determine the importance of RAGE-mediated cellular activation to human chronic diseases, it represents a novel receptor-ligand system potentially impacting on a range of patho-physiologic conditions, such as diabetes, inflammation, neurodegenerative disorders, and tumors.

Based on the link between protein glycation and the development of the health complications associated with diabetes and normal aging, it was hypothesized that inhibition of the protein glycation and the formation of AGEs in vivo may prevent or retard the development of the implicated health complications. Several studies in animal diabetic models have confirmed that the inhibition of protein glycation in vivo does indeed ameliorate diabetic complications. This lead to a flurry of research activity to identify anti-glycation agents as potential drug candidates for the treatment of age- and diabetes-related complications. Some of the major health complications that are retarded when protein glycation is inhibited in vivo include nephropathy, neuropathy, retinopathy, and cardiovascular dysfunction.

Aminoguanidine (AG) is presently the leading compound as an anti-glycation agent to prevent AGEs formation, and it is under clinical trial as a drug for the treatment of diabetic nephropathy and other diabetes-related complications (reviewed by Ulrich et al., Recent Prog. Horm. Res. 56, 1-21 (2001)). AG does not prevent the initial conjugation of proteins and reducing sugars to form a Schiff base and the subsequent rearrangement to Amadori products. Instead, it reacts with α-dicarbonyls such as 1-amino-1,4-dideoxyosone, glucosone, and glyoxal. The products of reaction between AG and α-dicarbonyl compounds are stable and do not participate in further reactions leading to formation of protein cross-links and AGEs. Another important AGE formation inhibitor under clinical trial for the treatment of diabetic complications is pyridoxamine (PM). The amino group of PM interacts with post-Amadori carbonyl intermediates and inhibits post-Amadori glycation reactions. PM also inhibits lipid oxidation by interacting with the keto-intermediate products of lipid auto-oxidation. Some of the inhibitors of AGEs formation reported in the literature are shown below.

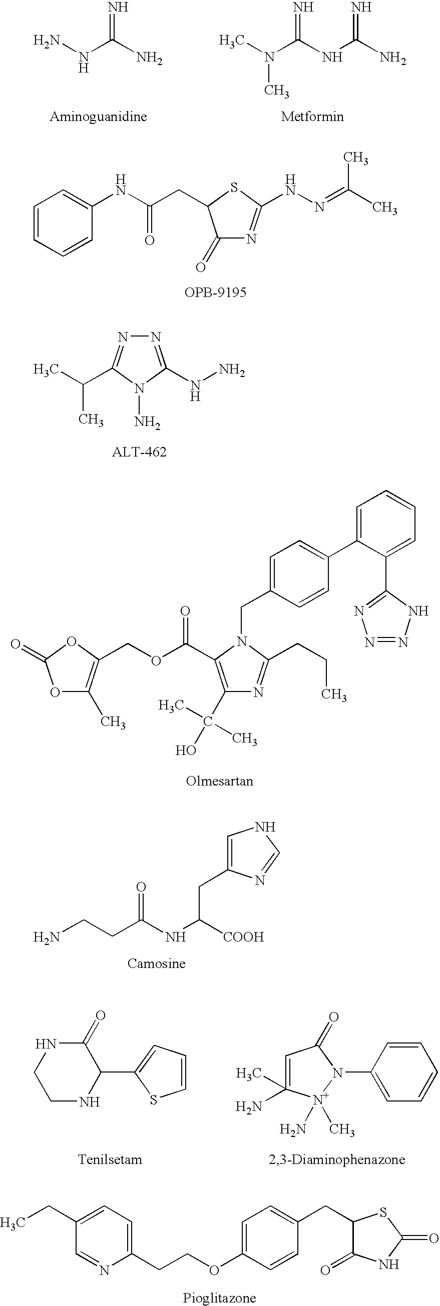

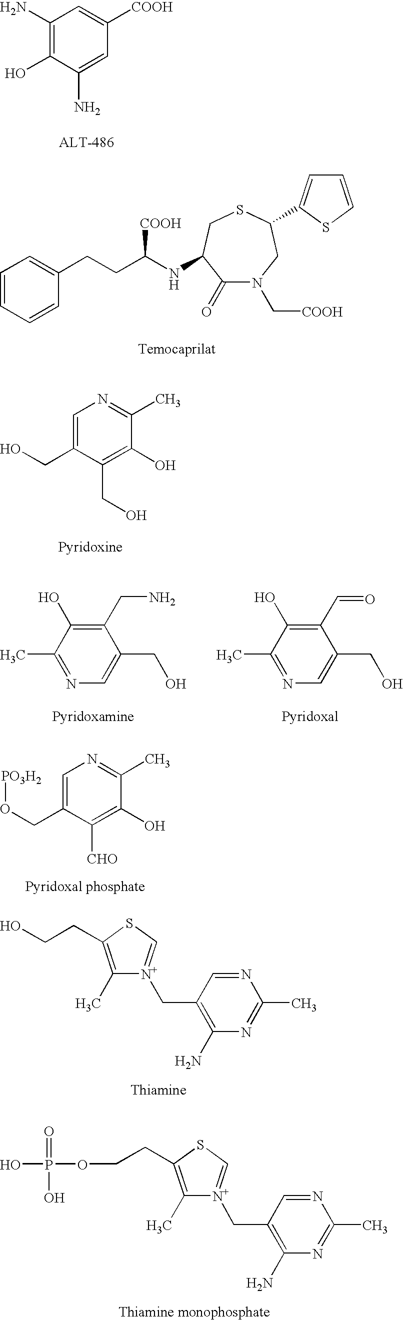

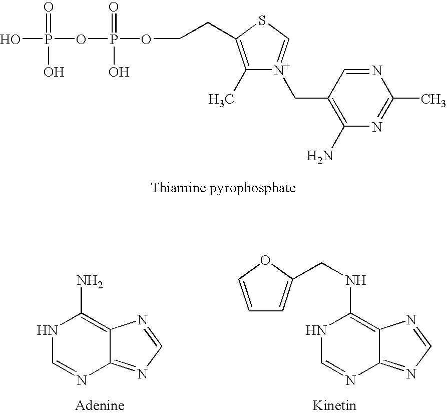

Some antioxidants, such as those shown below, are also known inhibitors of AGEs formation.

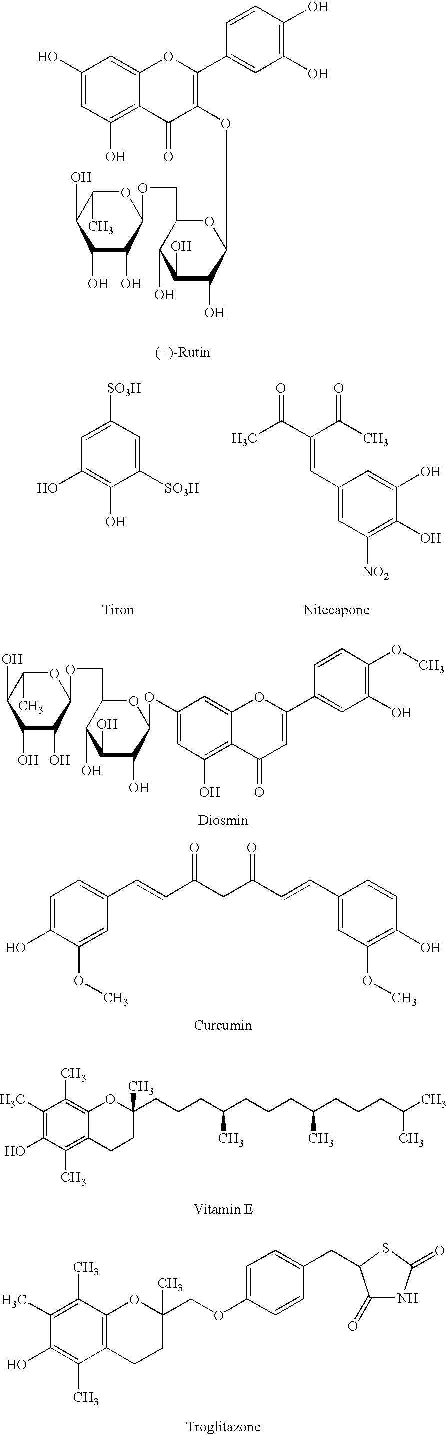

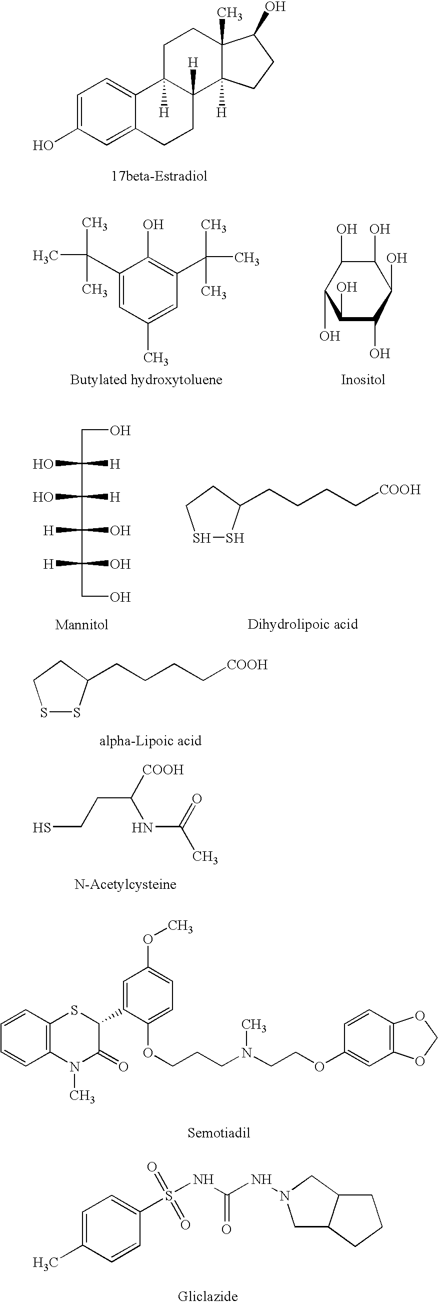

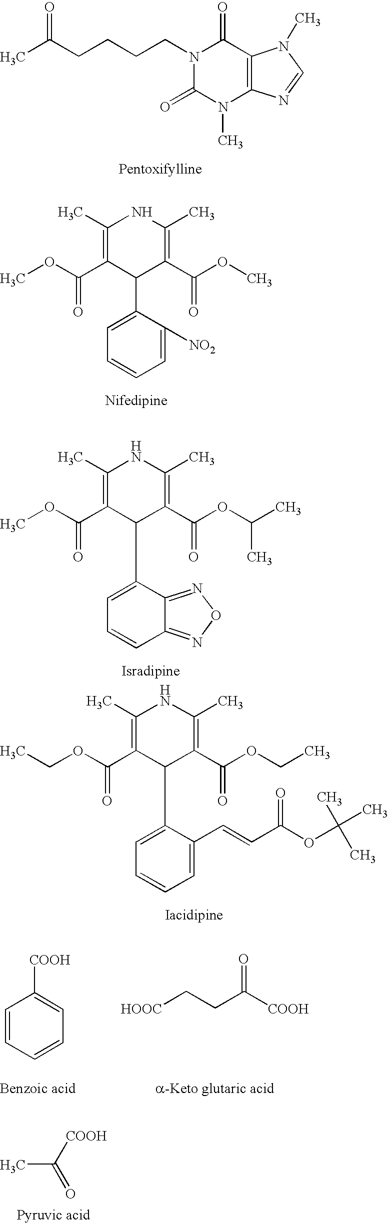

In addition to inhibiting the formation of AGEs, breaking down previously formed glycation-induced protein-protein cross-links has also been shown to ameliorate diabetes- and age-related complications in diabetic animal models. The reported compounds capable of breaking the glycation-induced protein-protein cross-links are thiazolium derivatives, exemplified by N-phenacylthiazolium bromide (PTB) and Alteon's ALT-711 (phenyl-4,5-dimethylthiazolium chloride). These compounds have been reported to reverse diabetes and age related myocardial stiffness and to improve cardiac function in diabetic rat models. AG, PM and ALT-711 are under clinical trials for the treatment of diabetic complications.

The level at which AG, the most investigated inhibitor of AGEs formation, shows therapeutic benefits in experimental diabetes (50 to 100 mg per kg body weight) is high and there is concern about possible side-effects under its long-term administration at those levels. A recent review of biological effects of AG noted that AG inhibits nitrous oxide synthase (which catalyses the synthesis of nitrous oxide from L-arginine), semicarbazide-sensitive amine oxidase (which catalyzes the deamination of methylamine and aminoacetone, leading to formation of cytotoxic formaldehyde and methylglyoxal, respectively) and diamine oxidase (which catalyses the degradation of bioactive diamines, such as histamine and putrescine). As a result, the therapeutic benefit of AG in ameliorating diabetes- and age-related health complications may not be due to its inhibition of glycation reaction (Nilsson, B. O., Inflamm. Res. 48, 509-515 (1999)).

Given the lack of insight into the mechanism of inhibition of protein glycation and its relationship to the prevention of diabetic complications, it is difficult to develop diabetic treatments based on anti-glycation agents. In view of this, and also in view of known disadvantages and limitations of prior art glycation inhibitors, it remains highly desirable to elucidate the details of the glycation mechanism and to develop effective, potent and safe inhibitors of protein glycation for the treatment of diabetes- and age-related health complications.

### SUMMARY OF THE INVENTION

The present invention provides novel anti-glycation agents. Some of the compounds identified as having this activity are novel and some are known. Those which are known may have other biological activities, but have not been previously shown to inhibit the glycation reaction and their anti-glycation properties have only been recognized by the present invention.

The anti-glycation compounds according to the present invention do not represent a single family of compounds, in the sense of sharing a common core chemical structure, and are characterized by a variety of chemical structures. The compounds of the invention may be classified based on either the presumed mechanism of their anti-glycation activity or on their chemical structure.

The anti-glycation compounds of the present invention are useful for the prevention or treatment of various age-, diabetes-, and smoking-related complications developed as a result the glycation reaction, such as neuropathy, nephropathy, vision impairment, or the loss of mechanical properties of collagenous tissues. Among compounds identified as having the anti-glycation activity, of special interest are epinephrine and its analogs, in particular D-epinephrine and its analogs, which were found to be particularly useful for the prevention or treatment of age-, diabetes- and smoking-related ocular pathologies.

### BRIEF DESCRIPTION OF THE DRAWINGS

FIG. 1 is a graph showing the inhibition of the Maillard fluorescence development by L-epinephrine. The concentration of L-epinephrine is plotted on X-axis in a log scale. The Y-axis represents the inhibition of the Maillard fluorescence development normalized by the fluorescence developed in the incubation of BSA (0.075 mM) for 100% inhibition and BSA (0.075 mM)+D-ribose (50 mM) for 0% inhibition.

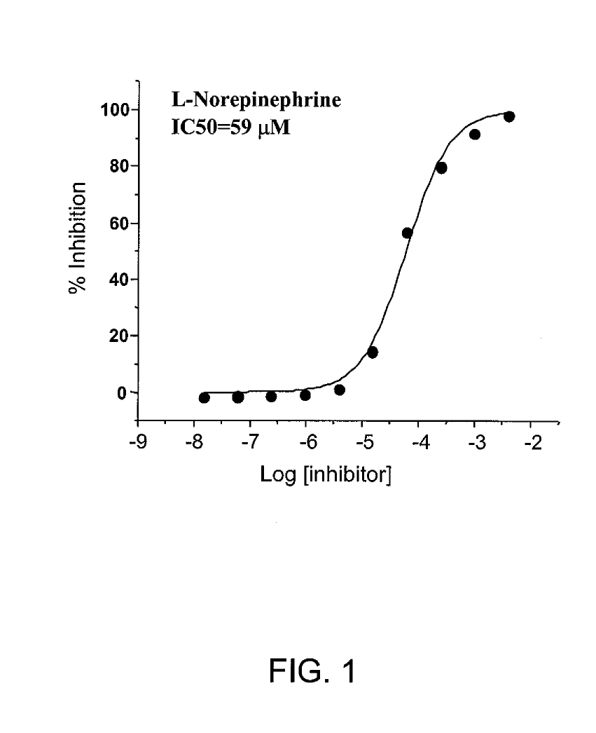

FIG. 2 illustrates the effects of anti-glycation agents (aminoguanidine and L-epinephrine) on the accumulation of glycation intermediates of lysozyme. Shown are mass spectra of lysozyme, lysozyme+D-ribose, lysozyme+D-ribose+L-epinephrine, and lysozyme+D-ribose+aminoguanidine incubated at 37° C. for 5 days.

### DETAILED DESCRIPTION OF THE INVENTION

The invention provides novel inhibitors of protein glycation and AGEs formation, many of them more potent and safer than inhibitors known in the prior art. These compounds have been identified from compound libraries by a high throughput screening assay. The mechanism of inhibition of the compounds so identified was then studied and a number of their structural analogs were synthesized, to develop lead candidates for the treatment of age-, diabetes-, and smoking-related complications.

During the glycation reaction between proteins and reducing sugars, a specific fluorescence with excitation and emission wavelength of 370 nm and 440 nm, respectively, is observed. This fluorescence, commonly referred to as Maillard fluorescence, is attributed to the formation of heterocyclic aromatic ring structures (both free and protein-bound) which constitute AGEs. A Maillard fluorescence-based assay was developed and optimized for screening compound libraries for chemical compounds that are able to inhibit the formation of AGEs. The assay was based on the progressive development of the characteristic Maillard fluorescence (370 nm Ex and 440 nm Em) during the progress of the glycation reaction.

The assay involved incubating together bovine serum albumin (BSA), D-ribose and a candidate anti-glycation agent (assay compound) using a microtitre plate (96 wells) at 37° C. in a closed system. Positive control (100% inhibition of the Maillard fluorescence formation or no Maillard fluorescence formation) consisted of wells with only BSA. Negative control (0% inhibition of the Maillard fluorescence formation) consisted of wells with BSA+D-ribose. The final assay volume was 200 μl and each assay well contained 0.075 mM BSA, and 50 mM D-ribose. Compounds were assayed at 3 different concentration levels (0.003, 0.03, and 0.3 mg/mL) to determine the effect of concentration on inhibition. Samples were incubated for 5 days.

Assay compounds that inhibited more than 30% of the AGEs fluorescence formation observed for the negative control were selected as possible anti-glycants for further studies. In order to eliminate false positives due to fluorescence quenching by the assay compounds, compounds that showed positive results were further subjected to a Maillard fluorescence-quenching test. In this test, the selected compounds were incubated with previously glycated BSA that had already developed Maillard fluorescence. The potency of the compounds that showed fluorescence quenching was further analyzed by separating the glycated BSA from the fluorescence quenching assay compound and low molecular weight degradation products on reverse phase (C-18) high performance liquid chromatography (RP-HPLC) column and quantitatively analyzing the Maillard fluorescence of the glycated BSA. After 5 days of incubation, all Maillard fluorescence was associated with BSA, with no Maillard fluorescence detected for the low molecular weight degradation products.

Further experiments were conducted on the selected compounds to determine the concentration at which they inhibited 50% of the development of Maillard fluorescence over a 5-day incubation period (IC₅₀). This was done by incubating selected anti-glycants at 10 different concentration levels in the range from 1 μM to 260 mM, from 0.25 μM to 66 mM, or from 0.015 μM to 4 mM with 0.075 mM BSA and 50 mM D-ribose under the earlier-described assay conditions. The Maillard fluorescence was directly measured from the reaction solution, except for the anti-glycants that quenched the Maillard fluorescence, which were analyzed by the earlier-described HPLC method. The IC₅₀ values of the tested anti-glycation agents are summarized in Table 1.

TABLE 1
IC₅₀ values of anti-glycation agents.

| | | | Un- | |
|---|---|---|---|---|
| | | | reacted | |
| | Mol. | | amino | Suppression |
| | mass | IC₅₀ | group | of BSA-BSA |
| Compound | (Da)^{a)} | (μM)^{b)} | (%)^{c)} | cross-links^{d)} |
| Acacetin | 284.07 | 510 | 67 | |
| Aclarubicin | 811.34 | 2500 | NA | |
| Adrenalone | 181.07 | 57 | 45 | |
| Aklomide | 200.00 | 350 | | |
| Aminitrozole | 187.01 | 82 | | |
| 2-Amino-5-nitropyrimidine | 140.03 | 810 | NA | |
| 4-Amino-salicylic acid | 153.04 | >4000 | | |
| Apigenin | 270.05 | 130 | | |
| Aristolochic acid | 341.05 | >4000 | NA | |
| Azathioprine | 277.04 | 400 | | |
| Baicalein | 270.05 | 49 | 48 | |
| BHA | 180.12 | >4000 | NA | |
| Biochanin A | 284.07 | 290 | | |
| Bopindolol | 380.21 | 170 | | |
| Botran | 205.96 | >4000 | NA | |
| Broxyquinoline | 300.87 | 390 | 67 | |
| Carbazochrome | 236.09 | 3500 | NA | |
| S(−)-Carbidopa | 226.10 | 140 | 89 | |
| beta-Carotene | 536.44 | 630 | | |
| (+)-Catechin | 290.08 | 19 | 66 | |
| 2-Chloro-4-nitrophenol | 172.99 | 3500 | NA | |
| Chloroxine | 212.97 | 1400 | NA | |
| Chloramphenicol | 322.01 | 1300 | NA | |
| Chloramphenicol palmitate | 560.24 | >4000 | NA | |
| Chrysin | 254.06 | 2100 | NA | |
| Cloxiquine | 179.01 | 440 | | |
| Corbadrine | 183.09 | 52 | | |
| Curcumin | 368.13 | 1100 | NA | |
| L-Cysteine ethyl ester | 149.05 | >4000 | NA | |
| Daidzein | 254.06 | >4000 | NA | |
| Dantrolene | 314.07 | 100 | 38 | |
| Demeclocycline | 464.10 | 19 | 69 | + |
| 3,4-Dihydroxy-1-[(α- | 225.14 | 78 | | |
| amino-β- | | | | |
| methoxy)ethyl]benzene | | | | |
| 3,4- | 138.03 | 120 | 32 | |
| Dihydroxybenzaldehyde | | | | |
| 3′.4′-Dihydroxyflavone | 254.06 | 2600 | NA | |
| 3,4-Dihydroxy-1-[(α- | 225.14 | 78 | | |
| isopropylamino-β- | | | | |
| methoxy)ethyl]benzene | | | | |
| 3,4-Dihydroxy-1-[α-(1- | 301.17 | 350 | | |
| methyl-3-phenyl- | | | | |
| propylamino)-β- | | | | |
| hydroxyethyl]benzene | | | | |
| 2,6-Diiodo-4-nitrophenol | 390.82 | 1300 | NA | |
| 3,5-Diiodo-L-tyrosine | 432.87 | 1500 | NA | |
| Dimetridazole | 141.05 | 460 | | |
| Dinitolmide | 225.04 | 340 | | |
| 3,5-Dinitrobenzamide | 211.02 | 780 | NA | |
| L-DOPA | 197.07 | 75 | 60 | * |
| Dopamine | 153.08 | 62 | 60 | * |
| Doxycycline | 444.15 | 33 | | |
| Ebselen | 274.98 | >4000 | NA | |
| Ellagic acid | 302.01 | 40 | 52 | |
| Emodin | 270.05 | 3000 | NA | |
| (−)-Epicatechin | 290.08 | 58 | 57 | |
| (−)-Epigallocatechin gallate | 458.08 | 48 (max. | 58 | |
| | | inhbition | | |
| | | 55%) | | |
| D,L-Epinephrine | 183.09 | 15 | | |
| L-Epinephrine | 183.09 | 18 | 69 | ++ |
| Eriodictyol | 288.06 | 250 | 43 | |
| Etanidazole | 214.07 | 450 | | |
| Evans blue | 872.05 | 3700 | NA | |
| Flutamide | 276.07 | 1300 | NA | |
| Fumaric acid | 116.01 | 2500 | NA | |
| Furaltadone | 324.11 | 41 (max. | 60 | |
| | | inhibition | | |
| | | 30%) | | |
| Furazolidone | 225.04 | 33 (max. | | |
| | | inhibition | | |
| | | 38%) | | |
| Galangin | 270.05 | 110 | 64 | |
| Genistein | 270.05 | 450 | | |
| Gossypol | 518.19 | 110 | 23 | |
| 6-Hydroxydopamine | 169.07 | 95 (max. | | |
| | | inhibition | | |
| | | 65%) | | |
| Hydroquinone | 110.04 | 250 | | |
| 8-Hydroxyquinoline | 145.05 | 95 | 55 | + |
| 8-Hydroxyquinoline-5- | 225.01 | 560 | 76 | ++ |
| sulfonic acid | | | | |
| Hydroxyurea | 76.03 | >4000 | NA | |
| Ifenprodil | 325.20 | >4000 | NA | |
| Indomethacin | 357.08 | 310 | 54 | |
| Isoetharine | 239.15 | 25 | 70 | + |
| D-Isoproterenol | 211.12 | 21 | 68 | + |
| L-Isoproterenol | 211.12 | 18 | 73 | + |
| Kaempferide | 286.05 | 280 | 49 | |
| Kojic acid | 142.03 | >4000 | NA | |
| Lapachol | 242.09 | 320 | 53 | |
| Lawsone | 174.03 | 190 | 44 | |
| Luteolin | 286.05 | 60 | 60 | |
| L-α-Methyl-DOPA | 211.08 | 73 | 47 | * |
| α-(1-Methyl-3-phenyl- | 299.15 | 890 | | |
| propylamino)-3,4- | | | | |
| dihydroxyacetophenone | | | | |
| Metronidazole | 171.06 | 660 | | |
| Minocycline | 457.18 | 46 | 59 | − |
| Mitoxantrone | 444.20 | 630 | 50 | + |
| Myricetin | 318.04 | 54 (max. | | |
| | | inhibition | | |
| | | 64%) | | |
| Naloxonazine | 650.31 | 1700 | NA | |
| Naringenin | 272.07 | 220 | | |
| Nicardipine | 479.21 | >4000 | NA | |
| Nifuroxazide | 275.05 | 150 | | |
| Nimesulide | 308.05 | 350 | 69 | |
| Nimodipine | 418.17 | >4000 | NA | |
| Nitrofurantoin | 238.03 | 44 (max. | | |
| | | inhibition | | |
| | | 40%) | | |
| Nitrofurazone | 198.04 | 46 (max. | | |
| | | inhibition | | |
| | | 50%) | | |
| 2-Nitroimidazole | 113.02 | >4000 | NA | |
| Nitrophenide | 307.99 | 4000 | NA | |
| p-Nitrophenol | 139.03 | >4000 | NA | |
| Nitroxoline | 190.04 | 300 | 76 | |
| D-Norepinephrine | 169.07 | 66 | 67 | + |
| L-Norepinephrine | 169.07 | 59 | 70 | + |
| Nylidrin | 299.19 | 1800 | NA | |
| Ornidazole | 219.04 | 570 | 60 | |
| Orthoform | 167.06 | 2100 | NA | |
| Oxantel | 216.13 | 600 | 55 | + |
| 5,7,3′,4′,5′- | 302.04 | 210 | 54 | |
| Pentahydroxyflavone | | | | |
| Phenidone | 162.08 | 1300 | NA | |
| Phenol red | 354.06 | 1800 | NA | |
| Piroxicam | 331.06 | 730 | | |
| Propyl gallate | 212.07 | 330 | 28 | |
| Purpurogallin | 220.04 | 76 | 31 | |
| Pyrithioxin | 127.01 | >4000 | | |
| Quercetin | 302.04 | 130 | | |
| Ranitidine | 314.14 | 1100 | NA | |
| RCL R70,335-4 | 360.08 | 230 | | |
| Rifampicin | 822.41 | 120 | 67 | + |
| Ritodrine | 287.15 | >4000 | NA | |
| Ronidazole | 200.05 | 950 | NA | |
| Roxarsone | 262.94 | 230 | | |
| Silibinin | 482.12 | 750 | NA | |
| Sinomenine | 329.16 | >4000 | NA | |
| Sulfasalazine | 398.07 | 55 | 64 | |
| Synephrine | 167.09 | >4000 | NA | |
| Taxifolin | 304.06 | 100 | | |
| Terbutaline | 225.14 | >4000 | NA | |
| Tetracycline | 444.15 | 11 | 48 | |
| 7,8,3′,4′- | 286.05 | 150 | 56 | |
| Tetrahydroxyflavone | | | | |
| (±)-Tetrahydropapaveroline | 287.12 | 90 | 51 | |
| Tinidazole | 247.06 | 700 | | |
| 3,3′,4′-Trihydroxyflavone | 270.05 | 1800 | NA | |
| 5,3′,4′-Trihydroxyflavone | 270.05 | 1600 | NA | |
| 5,7,2′-Trihydroxyflavone | 270.05 | 220 | 53 | |
| 6,3′,4′-Trihydroxyflavone | 270.05 | >4000 | NA | |
| 6,7,3′-Trihydroxyflavone | 270.05 | 940 | | |
| Trolox C | 250.12 | 470 | | |
| Trypan blue | 872.05 | 240 | 60 | + |
| Vitamin K5 | 173.08 | 90 | 51 | |
| Aminoguanidine | 74.06 | 345 | 68 | + |

^{a)}monoisotope molecular mass;
^{b)}the IC₅₀ values determined by the HPLC method are underlined (maximum percentage of the inhibition is also listed if it is significantly lower than 100%);
^{c)}percentage of free primary amino group on BSA after 5 days of incubation at 37° C. In the absence of the anti-glycation agent, 64% of the primary amino groups were unreacted;
^{d)}(++, strongly suppressed), (+, suppressed), (−, no suppression), (*, moderate acceleration of the cross-linking).

The effect of anti-glycation agents on the accumulation of glycated forms (glycoforms) of protein was evaluated by using electrospray mass spectrometry (ESI-MS). As the molecular mass of BSA was too large to monitor the small mass changes of its glycoforms, lysozyme was incubated with D-ribose and anti-glycants. Lysozyme and its glycoforms were isolated by RP-HPLC and analyzed by mass spectrometry.

To further evaluate the effects of the identified anti-glycation agents on protection of amino groups of proteins, the fluorescamine assay (Yeboah F. et al., J. Agric. Food Chem. 48, 2766-2774 (2000)) was performed on mixtures of BSA and D-ribose incubated in the presence and absence of the identified anti-glycation agents, to determine the number of lysine residues of BSA glycated during the incubation. The observed effects of the studied anti-glycation agents vary. Some of them, such as L-isoproterenol protect the amino group against glycation, whereas some other, such as 3,4-dihydroxybenzaldehyde, seem to accelerate the modification of the amino groups. The percentage of the amino group unreacted after 5 days of incubation at 37° C. is shown in Table 1.

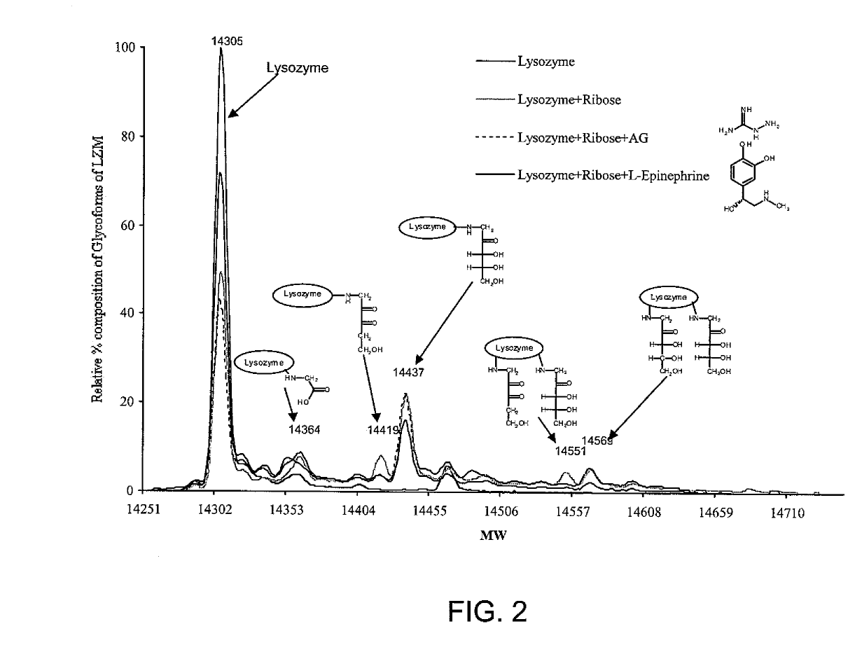

FIG. 2 shows the mass spectrometric profile of the glycoforms of lysozyme formed during the incubation in the absence and presence of anti-glycation agents. When lysozyme was incubated alone with D-ribose, glycoforms of lysozyme with up to two covalently bound D-ribose molecules were observed in the glycation mixture. In the presence of AG, the amount of glycoform of (AP-H₂O) was reduced whereas the amount AP was not. The presence of L-epinephrine reduced the amounts of both AP and (AP-H₂O) glycoforms. These results clearly indicate that each anti-glycation agent has different effects on the glycation pathways and on the amount of glycoforms. The amounts of unreacted lysozyme and the glycoforms for various anti-glycants are listed in Table 2.

TABLE 2
Peak areas as % of the total area under peaks of all glycoforms
measured by mass spectrometry. The molecular masses shown in the top row
correspond to peaks of unmodified lysozyme (14,305 Da), lysozyme with one
carboxymethylated Lys residue (14,364 Da), lysozyme + D-ribose − 3H₂O (14,401 Da),
lysozyme + D-ribose − 2H₂O (14,419 Da), lysozyme + D-ribose − H₂O (14,437 Da),
lysozyme + D-ribose − H₂O with one carboxymethylated Lys residue (14,496 Da),
lysozyme + 2D-ribose − 6H₂O (14,515 Da), lysozyme + 2D-ribose − 5H₂O (14,533 Da),
lysozyme + 2D-ribose − 4H₂O (14,515 Da), lysozyme + 2D-ribose − 3H₂O (14,551 Da),
and lysozyme + 2D-ribose − 2H₂O (14,569 Da), respectively.

| | 14,305 | 14,364 | 14,401 | 14,419 | 14,437 |
|---|---|---|---|---|---|
| Anti-glycation agents | (%) | (%) | (%) | (%) | (%) |
| Negative control without D-ribose and | 100 | 0 | 0 | 0 | 0 |
| anti-glycation agent | | | | | |
| Positive control without anti-glycation | 51.6 | 4.8 | 1.4 | 7.0 | 21.3 |
| agent | | | | | |
| Acacetin | 51.6 | 2.3 | 0.0 | 7.3 | 24.3 |
| Adrenalone | 59.3 | 4.8 | 1.7 | 2.5 | 17.2 |
| 2-Amino-5-nitropyrimidine | 52.9 | 3.6 | 0.0 | 7.7 | 23.0 |
| Baicalein | 94.1 | 0.0 | 0.0 | 0.0 | 5.9 |
| Broxyquinoline | 42.2 | 2.5 | 4.9 | 7.2 | 21.0 |
| S(−)-Carbidopa | 67.9 | 3.7 | 2.0 | 3.2 | 18.4 |
| (+)-Catechin | 68.9 | 2.0 | 0.0 | 4.0 | 19.7 |
| Demeclocycline | 75.2 | 4.2 | 1.9 | 3.0 | 13.7 |
| 3,4-Dihydroxybenzaldehyde | 78.6 | 0.0 | 0.0 | 3.1 | 10.9 |
| Dinitolmide | 51.7 | 3.0 | 0.0 | 7.4 | 22.6 |
| L-DOPA | 64.7 | 3.9 | 1.8 | 2.1 | 21.1 |
| Dopamine | 79.2 | 3.3 | 1.3 | 1.6 | 13.4 |
| Ellagic acid | 83.1 | 1.1 | 0.0 | 1.2 | 14.6 |
| (−)-Epicatechin | 76.3 | 1.8 | 0.0 | 2.6 | 17.6 |
| (−)-Epigallocatechin gallate | 69.1 | 2.0 | 0.0 | 2.6 | 20.9 |
| L-Epinephrine | 74.5 | 4.8 | 0.8 | 2.0 | 15.3 |
| Eriodictyol | 66.7 | 1.9 | 0.4 | 5.0 | 20.2 |
| Furaltadone | 60.6 | 0.9 | 0.0 | 6.3 | 23.9 |
| Furazolidone | 56.8 | 2.2 | 0.0 | 6.6 | 22.7 |
| Galangin | 48.0 | 4.1 | 0.8 | 10.2 | 20.2 |
| Gossypol | 58.4 | 1.0 | 0.0 | 6.0 | 23.9 |
| 8-Hydroxyquinoline | 67.8 | 4.2 | 5.8 | 3.6 | 9.0 |
| 8-Hydroxyquinoline-5-sulfonic acid | 58.0 | 0.7 | 2.0 | 3.2 | 27.9 |
| Indomethacin | 58.0 | 5.1 | 0.5 | 7.5 | 18.6 |
| Isoetharine | 73.4 | 3.9 | 1.3 | 1.6 | 14.3 |
| D-Isoproterenol | 76.2 | 4.7 | 0.8 | 1.8 | 13.7 |
| L-Isoproterenol | 67.0 | 5.3 | 2.5 | 2.3 | 16.5 |
| Kaempferide | 46.7 | 2.5 | 5.4 | 8.9 | 18.5 |
| Lapachol | 58.6 | 7.9 | 0.4 | 7.8 | 17.0 |
| Lawsone | 74.3 | 7.5 | 0.0 | 4.9 | 9.5 |
| Luteolin | 51.4 | 1.1 | 2.1 | 6.3 | 21.2 |
| L-α-Methyl-DOPA | 69.4 | 3.5 | 2.1 | 1.5 | 18.3 |
| Minocycline | 75.0 | 3.8 | 2.7 | 1.8 | 7.6 |
| Mitoxantrone | 49.8 | 1.6 | 1.9 | 6.5 | 25.2 |
| Myricetin | 78.9 | 1.6 | 0.0 | 1.8 | 17.7 |
| Naringenin | 56.8 | 0.1 | 0.0 | 7.5 | 24.4 |
| Nimesulide | 49.8 | 3.4 | 0.0 | 7.6 | 22.6 |
| Nitrofurazone | 60.8 | 1.6 | 0.0 | 5.8 | 21.4 |
| Nitroxoline | 66.7 | 4.2 | 3.3 | 7.8 | 11.0 |
| D-Norepinephrine | 77.3 | 5.0 | 1.9 | 1.9 | 12.1 |
| L-Norepinephrine | 75.9 | 5.3 | 2.3 | 2.4 | 12.0 |
| Ornidazole | 54.2 | 3.2 | 0.0 | 6.7 | 23.4 |
| Oxantel | 57.7 | 5.4 | 3.7 | 5.6 | 17.4 |
| 5,7,3′,4′,5′-Pentahydroxyflavone | 55.0 | 2.2 | 0.2 | 8.6 | 20.6 |
| Propyl gallate | 86.6 | 4.4 | 0.0 | 1.3 | 7.7 |
| Purpurogallin | 67.9 | 0.0 | 0.0 | 4.1 | 22.2 |
| Rifampicin | 64.7 | 5.4 | 2.9 | 4.1 | 15.8 |
| Roxarsone | 62.4 | 0.0 | 0.0 | 3.1 | 27.0 |
| Sulfasalazine | 47.2 | 2.9 | 2.7 | 8.2 | 20.6 |
| Tetracycline | 89.3 | 0.0 | 0.0 | 1.6 | 9.1 |
| 7,8,3′,4′-Tetrahydroxyflavone | 76.2 | 2.0 | 0.0 | 3.0 | 15.9 |
| (±)-Tetrahydropaveroline | 68.9 | 3.9 | 2.1 | 2.8 | 17.6 |
| 5,7,2′-Trihydroxyflavone | 46.7 | 5.8 | 1.6 | 9.3 | 19.8 |
| 6,3′,4′-Trihydroxyflavone | 56.4 | 2.0 | 0.3 | 8.8 | 21.5 |
| Trypan blue | 49.8 | 3.7 | 3.0 | 5.6 | 20.2 |
| Vitamin K5 | 75.1 | 4.9 | 0.0 | 3.3 | 12.2 |
| Aminoguanidine | 54.5 | 3.2 | 2.1 | 2.6 | 26.2 |
| | 14,496 | 14,515 | 14,533 | 14,551 | 14,569 |
| Anti-glycation agents | (%) | (%) | (%) | (%) | (%) |
| Negative control without D-ribose and | 0 | 0 | 0 | 0 | 0 |
| anti-glycation agent | | | | | |
| Positive control without anti-glycation | 1.9 | 0.5 | 1.1 | 3.7 | 4.2 |
| agent | | | | | |
| Acacetin | 0.0 | 0.9 | 1.8 | 3.7 | 5.3 |
| Adrenalone | 2.2 | 1.5 | 1.3 | 1.3 | 3.3 |
| 2-Amino-5-nitropyrimidine | 0.0 | 0.9 | 2.0 | 3.8 | 4.7 |
| Baicalein | 0.0 | 0.0 | 0.0 | 0.0 | 0.0 |
| Broxyquinoline | 1.5 | 1.7 | 3.2 | 4.7 | 5.7 |
| S(−)-Carbidopa | 0.9 | 0.6 | 0.8 | 2.4 | 0.0 |
| (+)-Catechin | 0.0 | 0.0 | 1.2 | 1.4 | 2.8 |
| Demeclocycline | 0.0 | 0.0 | 0.0 | 0.0 | 1.4 |
| 3,4-Dihydroxybenzaldehyde | 1.5 | 1.2 | 0.0 | 3.0 | 1.8 |
| Dinitolmide | 0.0 | 1.3 | 2.6 | 3.6 | 4.9 |
| L-DOPA | 1.2 | 0.7 | 0.8 | 0.7 | 3.0 |
| Dopamine | 0.0 | 0.0 | 0.0 | 0.0 | 1.2 |
| Ellagic acid | 0.0 | 0.0 | 0.0 | 0.0 | 0.0 |
| (−)-Epicatechin | 0.0 | 0.0 | 0.0 | 0.0 | 1.7 |
| (−)-Epigallocatechin gallate | 0.0 | 0.0 | 1.7 | 0.8 | 2.9 |
| L-Epinephrine | 1.0 | 0.0 | 0.0 | 0.0 | 1.7 |
| Eriodictyol | 0.0 | 0.0 | 1.1 | 1.6 | 3.1 |
| Furaltadone | 0.0 | 0.0 | 1.1 | 2.7 | 4.5 |
| Furazolidone | 0.0 | 0.9 | 1.7 | 3.1 | 4.6 |
| Galangin | 0.0 | 1.0 | 2.2 | 5.2 | 4.3 |
| Gossypol | 0.0 | 0.7 | 2.5 | 2.9 | 4.7 |
| 8-Hydroxyquinoline | 1.7 | 1.7 | 1.6 | 1.5 | 2.3 |
| 8-Hydroxyquinoline-5-sulfonic acid | 0.0 | 0.5 | 1.0 | 0.9 | 5.2 |
| Indomethacin | 0.0 | 0.0 | 1.6 | 3.3 | 3.5 |
| Isoetharine | 1.4 | 0.9 | 0.9 | 0.7 | 1.6 |
| D-Isoproterenol | 0.6 | 0.0 | 0.5 | 0.2 | 1.4 |
| L-Isoproterenol | 1.4 | 0.9 | 1.0 | 0.8 | 2.2 |
| Kaempferide | 0.0 | 1.6 | 4.0 | 4.4 | 3.9 |
| Lapachol | 0.0 | 0.9 | 1.4 | 2.8 | 2.7 |
| Lawsone | 0.0 | 0.8 | 0.9 | 0.0 | 0.0 |
| Luteolin | 0.8 | 1.4 | 3.9 | 3.0 | 5.2 |
| L-α-Methyl-DOPA | 1.0 | 0.5 | 0.1 | 0.5 | 2.7 |
| Minocycline | 3.6 | 0.0 | 2.3 | 1.7 | 1.6 |
| Mitoxantrone | 1.0 | 0.8 | 1.3 | 3.6 | 5.9 |
| Myricetin | 0.0 | 0.0 | 0.0 | 0.0 | 0.0 |
| Naringenin | 0.0 | 0.0 | 1.8 | 3.1 | 4.9 |
| Nimesulide | 0.0 | 1.3 | 2.3 | 3.8 | 5.2 |
| Nitrofurazone | 0.0 | 1.1 | 2.4 | 2.8 | 4.2 |
| Nitroxoline | 1.0 | 1.2 | 1.2 | 2.1 | 1.4 |
| D-Norepinephrine | 0.7 | 0.0 | 0.0 | 0.0 | 1.0 |
| L-Norepinephrine | 0.9 | 0.0 | 0.0 | 0.0 | 1.2 |
| Ornidazole | 0.0 | 0.9 | 2.0 | 3.4 | 4.8 |
| Oxantel | 1.7 | 1.1 | 1.7 | 2.0 | 3.1 |
| 5,7,3′,4′,5′-Pentahydroxyflavone | 0.0 | 0.7 | 1.7 | 4.3 | 4.3 |
| Propyl gallate | 0.0 | 0.0 | 0.0 | 0.0 | 0.0 |
| Purpurogallin | 0.0 | 0.0 | 0.9 | 1.2 | 3.6 |
| Rifampicin | 1.5 | 0.9 | 1.0 | 1.4 | 2.2 |
| Roxarsone | 0.0 | 0.0 | 1.2 | 1.3 | 5.1 |
| Sulfasalazine | 1.5 | 1.3 | 2.1 | 4.7 | 4.7 |
| Tetracycline | 0.0 | 0.0 | 0.0 | 0.0 | 0.0 |
| 7,8,3′,4′-Tetrahydroxyflavone | 0.8 | 0.0 | 0.0 | 0.6 | 1.6 |
| (±)-Tetrahydropaveroline | 0.9 | 0.0 | 0.8 | 0.7 | 2.3 |
| 5,7,2′-Trihydroxyflavone | 0.0 | 1.7 | 2.3 | 4.8 | 4.3 |
| 6,3′,4′-Trihydroxyflavone | 0.0 | 0.6 | 1.6 | 4.0 | 4.0 |
| Trypan blue | 2.1 | 1.6 | 1.9 | 3.4 | 4.6 |
| Vitamin K5 | 0.0 | 0.0 | 1.6 | 1.3 | 1.6 |
| Aminoguanidine | 1.4 | 0.7 | 1.1 | 1.2 | 5.7 |

Additionally, protein cross-links were semi-quantitatively assessed by SDS PAGE gel chromatography. The results are shown in Table 1, where “++” indicates strong suppression of protein-protein cross-linking, “+” indicates moderate suppression, “−” indicates no effect, and “*” indicates moderate acceleration of the cross-linking.

The anti-glycation compounds according to the present invention do not represent a single family of compounds in the sense of sharing a common core chemical structure, but are characterized by a variety of chemical structures. The compounds of the invention can be broadly classified as anti-oxidants and those for which the anti-glycation mechanism is not clear.

Based on their chemical structure, several groups of anti-glycation compounds sharing common structural features can be identified.

1. Compounds of formula (I)

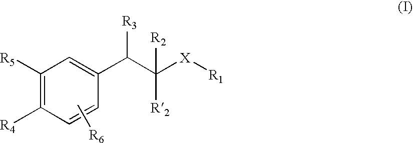

wherein:
X represents NR₇, wherein R₇ represents hydrogen atom or an acyl group derived from a linear or branched aliphatic acid or an aromatic acid,
R₁ represents hydrogen atom, NH₂, or a linear or branched C₁₋₅ alkyl which may be substituted with an aromatic group,
R₂ represents hydrogen atom, a linear or branched C₁₋₅ alkyl, or COOH group,
R′₂ represents hydrogen atom or a linear or branched C₁₋₅ alkyl group,
R₃ represents hydrogen atom, ═O, OR₈, SR₈, or NR₈R₉, wherein R₈ and R₉ represent hydrogen atom, a linear or branched C₁₋₅ alkyl, or an acyl group derived from a linear or branched aliphatic acid or an aromatic acid, provided that R₈ and R₉ are not both an acyl group,
R₄ and R₅ represent OR₁₀, or SR₁₀, wherein R₁₀ represents hydrogen atom or an acyl group derived from a linear or branched aliphatic acid or an aromatic acid,
R₆ represents hydrogen OR₁₀, or SR₁₀, wherein R₁₀ represents hydrogen atom or an acyl group derived from a linear or branched aliphatic acid or an aromatic acid.
   2. Compounds of formula (II)

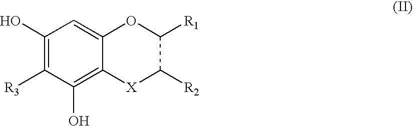

wherein:
R₁ represents H or an aromatic group which may be substituted with up to three hydroxyl groups,
R₂ represents H, OH, or an aromatic group which may be substitutes with hydroxyl groups, provided that at least one of R₁ and R₂ is an aromatic group,
R₃ represents H or OH,
X represents CH₂ or C═O,
   and wherein the dotted line represents single or double bond.
   3. Compounds of formula (III)

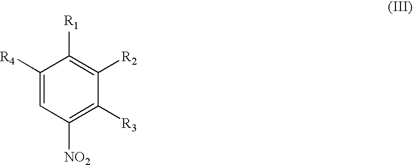

wherein:
R₁ represents H, OH, NH₂, NHR₅, an alkyl group which may be substituted with a polar group, or a halogen, wherein R₅ is an acyl derived from an aliphatic carboxylic acid or an aromatic sulfonic acid,
R₂ and R₄ represent independently H, halogen, or an aromatic ether group,
R₃ represents H or a polar group.
   4. Compounds of formula (IV)

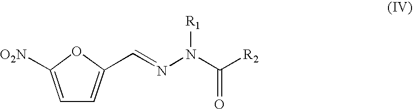

wherein:
R₁ represents H or an alkyl chain which may be connected to R₂,
R₂ represents C, N, O, or S, which atom may be substituted by an aromatic group or may be connected to R₁.
   5. Compounds of formula (V):

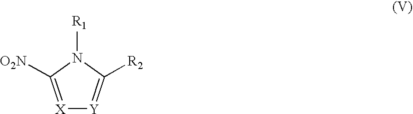

wherein:
R₁ and R₂ represent independently H or an alkyl chain which may be substituted with a polar group or groups, and wherein when one of X and Y is CH, the other one is N.
   6. Compounds of formula (VI)

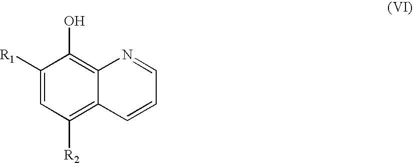

wherein:
R₁ and R₂ represent independently H or a polar group.
   7. Compounds of formula (VII)

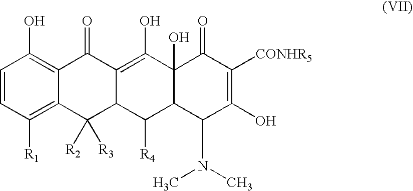

wherein:
R₁ represents hydrogen, chloro, or dimethylamino,
R₂ represents hydrogen or methyl,
R₃ represents hydrogen or hydroxy, or wherein R₂ and R₃ together represent ═CH₂,
R₄ represents hydrogen or hydroxy,
R₅ represents hydrogen, hydroxymethyl, or dialkylaminomethyl.

The anti-glycation compounds of the present invention are useful for the prevention or treatment of various age-, diabetes-, and smoking-related complications developed as a result of the glycation reaction, such as neuropathy, nephropathy, vision impairment, or the loss of mechanical properties of collagenous tissues. Among these applications, of particular interest for the present invention is the prevention of age-, diabetes-, and smoking-related ocular complications.

Human eye has a few natural antioxidants to prevent glycation. Pigment epithelium-derived factor (PEDF) in eye significantly inhibits AGE-induced reactive oxygen species generation (Yamaguchi et al., Biochem. Biophys. Res. Commun. 296, 877-882 (2002)). Reduced glutathione is a universal antioxidant and is presents in lens tissue in concentrations as high as 12-15 mM (Rose et al., Proc. Soc. Exp. Biol. Med. 217, 397-407 (1998)). Ascorbic acid is a major anti-oxidant that is present in millimolar concentrations in all ocular tissues (Richer, Int. Opthalmol. Clin. 40, 1-16 (2000)). Other natural ocular anti-glycants include antioxidant enzymes, such as superoxide dismutases, GSH peroxidase, GSH reductase, catalase, retinal reductase, and metallothionein, as well as ocular antioxidant cofactors, such as vitamins A, C, and E, and xanthophylls (Richer, supra). However, with age the above natural enzymatic protective systems become less functional, and the intake and absorption of requisite cofactor vitamins and minerals decrease (Richer, supra). Therefore, it remains highly desirable to develop effective, potent and safe inhibitors of protein glycation for the prevention of age-, diabetes-, and smoking-related ocular complications.

Among anti-glycation compounds of the present invention, of particular interest for ocular applications are compounds of formula (I), which can be seen as analogs of epinephrine. L-Epinephrine (also known as adrenaline) is a hormone secreted by the adrenal medulla of mammals, in response to low blood glucose levels, strenuous physical effort, and stress. Under these conditions, adrenaline causes a breakdown of glycogen to glucose in the liver, induces the release of fatty acids from adipose tissue, causes vasodilatation of the small arteries within muscles, and increases cardiac output. L-Epinephrine has a number of therapeutic applications, in particular for the treatment of anaphylactic shock, and is also used to treat certain types of glaucoma (high intra-ocular pressure).

In one preferred embodiment, the present invention provides a novel use of D-isoforms of epinephrine and its analogs, for preventing and treating age-, diabetes-, and smoking-related ocular complications. These compounds satisfy several criteria important for this application. First of all, the anti-glycation activity of the D-isoform of epinephrine and its analogs is high. Table 1 shows the IC₅₀ values of D-norepinephrine (IC₅₀=66 μM) and D-isoproterenol (IC₅₀=21 μM) that are essentially equivalent to those of L-norepinephrine (IC₅₀=59 μM) and L-isoproterenol (IC₅₀=18 μM), respectively. On this basis, it is reasonable to expect IC₅₀ values of D-epinephrine and its analogs as remaining in this range. Secondly, the adrenergic activity of the L-isoform, resulting in reducing the intra-ocular pressure, is insignificant for the D-isoform. The adrenergic activity of the D-isoform of epinephrine and its analogs is at least two orders of magnitude lower that that of the corresponding L-isoform (Patil et al., Pharmacol. Rev. 26, 323-392 (1974)). For the specific application of reducing the intra-ocular pressure, topical administration of up to 20% D-isoproterenol hydrochloride did not lower intra-ocular pressure in the human eye (Kass et al., Opthalmol. 15, 113-118 (1976)).

The D-isoform of epinephrine and its analogs is known to be safe for ocular administration. Various commercial preparations for the treatment of glaucoma contain D,L-epinephrine dipivalate (dipivefrin), which is a prodrug hydrolyzed to D,L-epinephrine after application to the eye. The liberated epinephrine contains equal amounts of the D- and L-isoform of epinephrine, of which only the adrenergically active L-isoform is relevant to the treatment of glaucoma. The D-isoform is inactive for this application, but its presence was proven to be safe. As preparations according to one preferred embodiment of the present invention contain only the D-isoform of epinephrine and its analogs, they are also safe for ocular applications.

Epinephrine is known to have the duration long enough for a reasonable frequency of administration, such as a twice-a-day administration. The duration of D,L-epinephrine was measured after topical administration of a 50 μL eye drop of 0.05% dipivefrin to rabbit's eye. The concentrations of D,L-epinephrine in choroid & retina were 2.96±1.11 μM, 3.76±0.37 μM, 2.19±0.39 μM, and 1.91±1.11 μM at 30 min, 1 hour, 3 hours and 6 hours, respectively, demonstrating the long duration of D,L-epinephrine in the eye (Wei et al., Invest. Opthalmol. Vis. Sci. 17, 315-321 (1978)).

It was also shown that epinephrine distributes at reasonably high concentrations in various ocular tissues. After application of a 50 μL drop of 0.05% dipivefrin to rabbit's eye, the following distribution of epinephrine was found after 6 hours: 2.78±0.39 μM in cornea, 0.28±0.08 μM in aqueous humor, 9.05±1.68 μM in iris, 3.71±0.67 μM in ciliary body, 1.91±1.11 μM in choroid and retina, 2.66±0.57 μM in sclera, <0.26 μM in lens and <0.026 μM in vitreous humors (Wei et al., supra). There is almost no variability in distribution among rabbits, cats, and monkeys (Kramer, Trans. Am. Opthalmol. Soc. 78, 947-982 (1980)). The concentrations in cornea, iris, ciliary body, choroid, retina, and sciera are comparable to IC₅₀ values of epinephrine and its analogs shown in Table 1. Moreover, the concentrations of intracellular reactive oxygen species required for glycation are drastically reduced by treatment with 1 μM noradrenaline. With EC₅₀ value of about 0.3 μM, noradrenaline is known to remarkably reduce oxidative stress related to glycation, and to promote long-term survival and function of dopaminergic neurons (Troadec et al., J. Neurochem. 79, 200-210 (2001)). In view of the above, D-epinephrine and D-enantiomers of its analogs, such as those represented by formula (I), may be particularly advantageously used for the prevention and treatment of ocular pathologies developed as a result of the glycation reaction.

For the use according to the invention, compounds of formula (I), in particular D-epinephrine and its analogs, can be used in the form of their physiologically tolerated salts, physiologically functional derivatives, or prodrugs. Preferred prodrugs or physiologically functional derivatives of compounds of formula (I) are those comprising at least one acyl group derived from a linear or branched aliphatic acid or an aromatic acid, wherein the acyl group acylates at least one of X, R₃, R₄, R₅, or R₆. Pivaloyl (trimethylacetyl) acyl group is particularly preferred.

Compositions for the ocular treatment according to the present invention may contain one or more compounds of formula (I), their physiologically tolerated salts, or physiologically functional derivatives, and may contain further active ingredients, such as an antimicrobial agent or agents, if required or appropriate. These compositions may be formulated in any dosage form suitable for topical ophthalmic delivery, such as solutions, suspensions, or emulsions. Of those, aqueous ophthalmic solutions are preferred. Other than the active ingredient(s), the compositions may further contain customary ophthalmic additives and excipients, such as a tonicity adjusting agent, a viscosity enhancing agent, or a surfactant.

### EXPERIMENTAL

### Materials

All synthetic products were purified using silica gel column chromatography with different solvents as eluents or by recrystallization from various solvents according to the procedures. The purity of compounds was established using an analytical Waters HPLC (Symmetry 3.5 by 50 mm C₁₈ reverse-phase column, gradient 5-60% acetonitrile in water, 0.1% TFA; flow rate 0.8 mL/min, 15 min, or Jones Chromatography 4.6 by 250 mm C₁₈ reverse-phase column, isocratic mode, 100% water, 0.1% TFA, flow rate 1 mL/min, 15 min). The compounds were characterized by mass spectrometry using an electrospray ionization mass spectrometer (ESI-MS) (Sciex API III mass spectrometer) and by ¹H NMR (Bruker-DRX-500 MHz).

### α-Isopropylamino-1-(2-hydroxyphenyl)ethanol hydrochloride

### α-isopropylamino-2-hydroxyacetophenone hydrochloride

Isopropylamine (1.18 g, 1.7 mL, 20 mmol) was slowly added dropwise to an ice-cooled solution of α-bromo-2-hydroxyacetophenone (2.15 g, 10 mmol) in anhydrous diethyl ether (25 mL). The reaction mixture was kept overnight at room temperature, then water (50 mL) was added. The separated organic phase was further washed with water (50 mL) and dried over anhydrous Na₂SO₄. Ether was removed and the remaining oil was treated with excess of hydrogen chloride in anhydrous diethyl ether to give a solid product.

Rt=3.73 min; MS [M+1]=230.9.

### α-isopropylamino-β-(2-hydroxyphenyl)ethanol hydrochloride

To a solution of α-isopropylamino-2-hydroxyacetophenone hydrochloride (0.465 g, 2 mmol) in methanol (25 mL), sodium borohydride (0.23 g, 60 mmol) was added in small portions. The reaction mixture was kept overnight at room temperature and the solvent was removed in vacuum. Water (30 mL) was added to the solid and stirred until all the inorganics were dissolved. The mixture was extracted with diethyl ether (30 mL) and the combined extracts were washed with water (20 mL), dried over anhydrous Na₂SO₄ and treated with excess of hydrogen chloride in anhydrous diethyl ether, with the product separating as a solid.

Rt=4.02 min; MS [M+1]=232.5;

NMR: 1H NMR (500 MHz, CD₃OD): δ(ppm) 1.28 (dd, J=6 Hz, 6H), 3.12 (m, J=5 Hz, 2H), 3.28 (s, 1H), 4.63 (m, J=7 Hz, 1H), 6.89 (d, J=8 Hz, 1H), 6.92 (d, J=10 Hz, 1H), 7.24 (d, J=7 Hz, 1H), 7.24 (d, J=7 Hz, 1H), 7.28 (d, J=7 Hz, 1H).

### α-(1-Methyl-3-phenyl-propylamino)-3,4-dihydroxyacetophenone hydrochloride

α-Chloro-3,4-dihydroxyacetophenone (1 g, 5.38 mmol) was dissolved in 10 mL of acetonitrile. 3-Amino-1-phenylbutane (0.87 mL, 5.38 mmol) was added and the mixture was stirred at room temperature for 4 hrs. The crude product precipitated from the reaction mixture and was filtered off. After washing with ether, the material was dissolved in 5 mL of 4N HCl and 10 mL of methanol. After filtration through decolorizing charcoal, the solution was evaporated in vacuum to give the desired hydrochloride.

Rt=4.89 min; MS [M+1]=300.1;

NMR: 1H NMR (500 MHz, CD₃OD): δ(ppm) 1.34 (t, J=6 Hz, 3H), 1.85 (d, J=40 Hz, 2H), 2.68 (m, J=7 Hz, 2H), 3.27 (m, J=12 Hz, 1H), 4.80 (q, J=12 Hz, 2H), 6.83 (d, J=8 Hz, 1H), 7.19 (d, J=10 Hz, 3H), 7.23 (s, 1H), 7.27 (d, J=7 Hz, 1H), 7.43 (d, J=11 Hz, 2H).

### 3,4-Dihydroxy-1-[α-(1-methyl-3-phenyl-propylamino)-β-hydroxyethyl]benzene hydrochloride

α-(1-methyl-3-phenyl-propylamino)-3,4-dihydroxyacetophenone hydrochloride was dissolved in 100 mL of methanol, Pd/C (0.1 g) was added and the mixture stirred at room temperature for 3 hrs under hydrogen bubbling. The reaction mixture was filtered, the filtrate evaporated in vacuum and the residue crystallized from ether yielding the desired hydrochloride salt.

Rt=5.28 min; MS: [M+1]=302.2;

NMR: 1H NMR (500 MHz, CD₃OD): δ(ppm) 1.34 (d, J=6 Hz, 3H), 1.45 (m, J=8 Hz, 2 H), 1.84 (d, J=40 Hz, 2H), 2.70 (m, J=7 Hz, 2H), 3.27 (m, J=12 Hz, 1H), 4.10 (q, J=12 Hz, 1H), 6.57 (d, J=8 Hz, 1H), 6.64 (d, J=7 Hz, 2H), 7.19 (d, J=10 Hz, 3H), 7.23 (s, 1H), 7.28 (d, J=7 Hz, 1H).

### 3,4-Dihydroxy-1-[α-isopropylamino-β-methoxy)ethyl]benzene

A mixture of isoproterenol hydrochloride (0.2 g, 0.81 mmol), SOCl₂ (1 ml), and a catalytic amount of dimethylformamide (DMF) was stirred in a 25 mL round bottom flask at 40° C. for 2 h. The resulting yellowish solution was evaporated to dryness and the residue crystallized from a mixture of acetone/methanol to yield the desired 3,4-dihydroxy-1-[α-isopropylamino-β-methoxy)ethyl]benzene as colorless crystals.

Rt=3.18 min; MS [M+1]=226;

NMR: 1H NMR (500 MHz, CD₃OD): δ(ppδ) 1.39 (t, J=6 Hz, 6H), 3.12 (q, J=10 Hz, 2H), 3.29 (s, 3H), 3.37 (q, J=5 Hz, 2H), 4.40 (q, J=9 Hz, 1H), 6.76 (d, J=7 Hz, 2H), 6.86 (s, 1H).

### 3,4-Dihydroxy-1-[(α-amino-β-methoxy)ethyl]benzene

A mixture of epinephrine (0.2 g, 1.09 mmol), SOCl₂ (1 mL) and a catalytic amount of DMF was stirred in a 25 mL round bottomed flask at 40° C. for 2 hrs. The resulting yellowish solution was then evaporated to dryness and the residue crystallized from a mixture of acetone/methanol to yield the desired product as colorless crystals.

Rt=1.85 min; MS [M+1]=198;

NMR: 1H NMR (500 MHz, CD₃OD): δ(ppm) 2.72 (s, 3H), 3.08 (q, J=10 Hz, 2H), 3.25 (s, 3H), 4.34 (q, J=9 Hz, 1H), 6.68 (d, J=7 Hz, 2H), 6.79 (s, 1H).

### D-norepinephrine dipivalate

### FMOC-D-norepinephrine

D-norepinephrine bitartrate (1 eq.), FMOC-succinamide (1 eq) and sodium bicarbonate (2 eq) were mixed in an acetonitrile-water mixture (9:1 ratio) and stirred vigorously for 18 hours. The insoluble part was filtered off and the solution was poured into 5% acetic acid. The suspension of FMOC-derivative in water was filtered off. The solid residue was washed two times with 5% acetic acid and three times with water and dried. The product was used further without purification (purity >95% according to HPLC)

Rt=8.9; MS [M+1]=392.

### FMOC-D-norepinephrine dipivalate

An equimolar mixture of FMOC-norepinephrine and 0.5 M sodium hydroxide was dissolved in water—DMF mixture (1:1) and after 5 minutes the solution was immediately mixed with a solution of 6 equivalents of pivalyl chloride in DMF. The resulting mixture was stirred for 15 minutes and was extracted three times with diethyl ether. The organic solvent was evaporated giving an oily residue containing a mixture of mono- and dipivalate of FMOC-norepinephrine. This was used without further purification.

Rt=11.9 min; MS [M+1]=560.

### D-norepinephrine dipivalate

A mixture of FMOC-D-norepinephrine dipivalate and monopivalate was dissolved in a solution of piperazine (20%) in DMF. After reacting for 20 minutes the solvents was evaporated under reduced pressure and the residue was purified by preparative HPLC giving the desired product.

Rt=7.5 min; MS [M+1]=338;

NMR: 1H NMR (500 MHz, CD₃OD): δ (ppm) 1.30 (s, 18H), 2.60 (q, J=6 Hz, 2H), 4.060 (q, J=6 Hz, 1H), 6.67 (s, 1H), 6.73 (d, J=7 Hz, 1H), 6.76 (d, J=7 Hz, 1H).

### D-isoproterenol dipivalate

### FMOC-D-isoproterenol

D-isoproterenol bitartrate (1 eq), FMOC-succinamide (1 eq) and sodium bicarbonate (2 eq) were mixed in an 1,4-dioxane-water mixture (9:1 ratio) and stirred vigorously for 18 hours. The insoluble part was filtered off and the solution was poured into 5% acetic acid. The suspension of FMOC-derivative in water was extracted three times with diethyl ether and the organic solvent was evaporated. The solid residue was washed with water-acetic acid mixture and dried. The product was used further without purification.

Rt=9.4 min; MS [M+1]=434.

### FMOC-D-isoproterenol dipivalate

An equimolar mixture of FMOC-isoproterenol and sodium carbonate was dissolved in water and the solution was immediately mixed with a solution of pivalyl chloride (3 eq.) in acetone. The resulting mixture was stirred until all traces of pivalyl chloride disappeared. Then the mixture was extracted three times with diethyl ether and the organic solvent evaporated giving an oily residue containing a mixture of mono- and dipivalate of FMOC-isoproterenol. This was used next without further purification.

Rt=11.2 min; MS [M+1]=602.

### D-isoproterenol dipivalate

A mixture of FMOC-D-isoproterenol dipivalate and monopivalate was dissolved in a solution of piperazine (20%) in DMF. After 20 minutes the solvents was evaporated under reduced pressure and the residue was purified by preparative HPLC to give the desired product.

Rt=8.00 min; MS [M+1]=380;

NMR: 1H NMR (500 MHz, CD₃OD): δ(ppm) 1.2 (d, J=10 Hz, 6H), 1.7 (s, 18H), 2.9 (q, J=5 Hz, 1H), 3.35 (q, J=10 Hz, 2H), 4.7 (q, J=9 Hz, 1H), 6.70 (d, J=7 Hz, 1H), 6.75 (d, J=7 Hz, 1H), 6.85 (s, 1H).

### D-3,4-Dihydroxy-1-[α-methylamino-β-hydroxy)ethyl]benzene hydrochloride (D-Epinephrine)

Dimethyl sulfate (0.1 ml, 10 eq) in 1 ml of methanol was added to a cooled mixture of D-norepinephrine bitartrate, (35.8 mg, 0.11 mmol), sodium hydroxide aqueous solution, 0.5 N (1 mL) and methanol (1 mL). The mixture was heated at 60° C. with stirring for 30 seconds. Then the reaction was stopped by adding hydrochloric acid, 1N (1 mL). D-epinephrine hydrochloride was purified by HPLC.

Rt=11.6 min.; MS [M+1]: 184;

NMR: 1H NMR (500 MHz, CD₃OD): δ (ppm) 2.72 (s, 3H), 3.77 (q, J=10 Hz, 2H), 4.23 (q, J=9 Hz, 1H), 6.74 (d, J=7 Hz, 1H), 6.76 (d, J=7 Hz, 1H), 6.86 (s, 1H).

L-Epinephrine (cat. No., 195166), azathioprine (cat. No. 191364), 2-chloro-4-nitrophenol (cat. No. 150635), furaltadone (cat. No. 158206), hydroquinone (cat. No. 150131), L-isoproterenol (cat. No. 195263), metronidazole (cat. No. 155710), minocycline (cat. No. 155718), nicardipine (cat. No. 190244), nimodipine (cat. No. 159803), ornidazole (cat. No. 155999), sulfasalazine (cat. No. 191144), terbutaline (cat. No. 156747), vitamin K5 (cat. No. 103284), S(−)-carbidopa (cat. No., 153757), D-isoproterenol (cat. No., 195263), 6-hydroxydopamine (cat. No. 153689), L-cysteine ethyl ester (cat. No., 101443), hydroxyurea (cat. No., 102023), emodin (cat. No., 190453), tetracycline (cat. No. 103011), ranitidine (cat. No., 153563), doxycycline (cat. No., 195044), piroxicam (cat. No., 156277), L-DOPA (cat. No., 101578), and L-α-methyl-DOPA (cat. No. 155517) were purchased from ICN. Isoetharine (cat. No., 13639), biochanin A (cat. No. D2016), 3,5-diiodo-L-tyrosine (cat. No. D0754), dimetridazole (cat. No. D4025), (−)-epigallocatechin gallate (cat. No. E4143), etanidazole (cat. No. E3016), flutamide (cat. No. F9397), fumaric acid (cat. No. F2752), furazolidone (cat. No. F9505), genistein (cat. No. G6649), gossypol (cat. No. G8761), mitoxantrone (cat No. M6545), nifuroxazide (cat. No. N2641), nimesulide (cat. No. N1016), nitrofurantoin (cat. No. N7878), 2-nitroimidazole (cat. No. N3882), oxantel (cat. No. 04755), phenidone (cat. No. P3441), phenol red (cat. No. P3532), ritodrine (cat. No. R0758), ronidazole (cat. No. R7635), silibinin (cat. No. S0417), daidzein (cat. No., D7802), pyrithioxin (cat. No. P7171), tyramine (cat. No., T2879), and L-ascorbic acid (cat. No., A2218) were purchased from Sigma. Methoxamine (cat. No. M-134), Dopamine (cat. No. D-019), corbadrine (cat. No., M-133), ifenprodil (cat. No. I-118), naloxonazine (cat. No. N-176), dantrolene (cat. No. D-145), and aminoguanidine (cat. No., A-199) were purchased from RBI. Synephrine (cat. No., 287237), D-norepinephrine (Cat. No. 40,745-3), Iapachol (cat. No. 142905), 4-amino-salicylic acid (cat. No. 856541), 2-amino-5-nitropyrimidine (cat. No. A70836), baicalein (cat. No. 46, 511-9), chloroxine (cat. No. D64600), dinitolmide (cat. No. 524417), kojic acid (cat. No. 22,046-9), nitrophenide (cat. No. N21006), nitroxoline (cat. No. 140325), RCL R70,335-4 (cat. No. R703354), and sinomenine (cat. No., 365602) were purchased from Aldrich. (+)-Catechin (cat. No. 22110), galangin (cat. No. 48291), indomethacin (cat. No. 57413), acacetin (cat. No. 00017), BHA (cat. No. 20021), beta-carotene (cat. No. 22040), chloramphenicol (cat. No. 23275), demeclocycline (cat. No. 30910), ellagic acid (cat. No. 45140), luteolin (cat. No. 62696), myricetin (cat. No. 70050), p-nitrophenol (cat. No. 73560), propyl gallate (cat. No. 48710), rifampicin (cat. No. 83907), Trypan blue (cat. No. 93590), and apigenin (cat. No. 10798) was purchased from Fluka. Botran (cat. No. 45435), and chloramphenicol palmitate (cat. No. 46109) was purchased from Riedel-de Haen. L-Norepinephrine (cat. No., 489350) Trolox C (cat. No. 648471), and aristolochic acid (cat. No. 182300) was purchased from CalBiochem. Adrenalone (cat. No., 6010), broxyquinoline (cat. No. 6948), 3,5-dinitrobenzamide (cat. No. 4991), 8-hydroxyquinoline (cat. No. 2743), 8-hydroxyquinoline-5-sulfonic acid (cat. No. 8268), naringenin (cat. No. 9834), orthoform (cat. No. 5687), and aminitrozole (cat. No. 1356) was purchased from Lancaster Synthesis. (−)-Epicatechin (cat. No., AC29194), 2,6-diiodo-4-nitrophenol (cat. No. AC16339), (±)-tetrahydropapaveroline (cat. No. AC22162), and lawsone (cat. No. AC12163) was purchased from Acros Organics. Ebselen (cat. No. E-1011) was purchased from A.G. Scientific. Taxifolin (cat. No., P-101), chrysin (cat. No. C005), curcumin (cat. No. C-004), eriodictyol (cat. No. 021111S), kaempferide (cat. No. K101), 5,7,3′,4′,5′-pentahydroxyflavone (cat. No. 22340), 7,8,3′,4′-Tetrahydroxyflavone (cat. No. T201), 3,3′,4′-trihydroxyflavone (cat. No. T601), 5,3′,4′-trihydroxyflavone (cat. No. T406), 6,7,3′-trihydroxyflavone (cat. No. 22336), 6,3′,4′-trihydroxyflavone (cat. No., T408), 5,7,2′-trihydroxyflavone (cat. No., T407) was purchased from INDOFINE Chemical Co. 3,4-Dihydroxybenzaldehyde (cat. No., A11558), Evans blue (cat. No. A16774), and aklomide (cat. No. A19702) was purchased from Alfa Aesar. Purpurogallin (cat. No. P0542), cloxiquine (cat. No. C0645), nitrofurazone (cat. No. N0200), quercetin (cat. No. P0042), roxarsone (cat. No. H0287), and carbazochrome (cat. No. A0176) was purchased from TCI. Bopindolol (cat. No. AR-100) was purchased from BIOMOL Research Lab. Inc. Tinidazole (cat. No. T1218) was purchased from Spectrum.

### Methods

### 1. High-Throughput Screening Assay of Anti-Glycation Agents

A Maillard fluorescence-based assay was developed and optimized for screening compound libraries for chemical compounds that are able to inhibit the formation of AGEs. The assay involved incubating BSA (0.075 mM protein concentration or 4.53 mM of Lys residue concentration) with D-ribose (50 mM) and a chemical compound (assay compound) (0.003, 0.03, and 0.3 mg/mL). Solutions were incubated in microtitre plates (96 wells) at 37° C. for 5 days in a closed system. (All incubation experiments were carried out in a closed system.) Positive control, i.e., 100% inhibition of the Maillard fluorescence formation (or 0% Maillard fluorescence formation) consisted of wells with only BSA. Negative control i.e., no inhibition of the Maillard fluorescence formation, consisted of BSA (0.075 mM) with D-ribose (50 mM). The final assay volume was 200 μL. Assay compounds that inhibited more than 30% of the AGEs fluorescence development were selected as possible anti-glycation agents for further studies.

In order to eliminate false positives due to fluorescence quenching by the assay compounds, compounds that showed positive results were further subjected to a Maillard fluorescence-quenching test. In this test, selected compounds with a concentration of 0.5 mg/mL were incubated with previously glycated BSA (0.075 mM) that had already developed Maillard fluorescence. After one hour incubation at 37° C., fluorescence readings were taken. Compounds that showed fluorescence quenching were analyzed separately, as described in the following section.

### 2. Determination of IC₅₀ of Selected Anti-Glycation Agents.

Further experiments were conducted on selected compounds that showed no fluorescence quenching, to determine their potency (IC₅₀). This was done by using 10 different concentration levels in 2 different concentration ranges, from 0.25 μM to 66 mM and from 15 nM to 4 mM. The experimental conditions were the same as those for the screening experiments.

The IC₅₀ values of the compounds that quenched the fluorescence of the glycated BSA were analyzed by on-line monitoring of the fluorescence of the glycated BSA separated from the fluorescence quenching assay compound by RP-HPLC. The elution profile was monitored by UV-diode array and by fluorescence (λₑₓ=370 nm, λₑₘ=440 nm). The fluorescence peak area of the glycated BSA was used as a measure of inhibition (%) by the anti-glycation agents after normalizing it with the peak areas of positive control (100% inhibition) and negative control (0% inhibition) as described above. The incubation conditions were the same as above.

### 3. Fluorescamine Assay

Fluorescamine assay (Yeboah F. et al., J. Agric. Food Chem. 48, 2766-2774 (2000)) was performed on incubated mixtures of BSA and D-ribose, with or without the identified anti-glycation agents. The mixtures contained BSA (0.075 mM protein concentration or 4.53 mM of Lys residue concentration) and D-ribose (50 mM). The final concentrations of the anti-glycation agents were adjusted to 16.8 times of the IC₅₀ values estimated in the earlier experiment. At these concentrations, most (statistically 98%) of the anti-glycation agents inhibit 80% or more of the Maillard fluorescence development. The fluorescamine assay determines the number of free lysine residues of BSA. In this experiment, the final volume of the incubation mixtures was 10 mL and the incubation time was 5 days at 37° C. Prior to the fluorescamine assay, the proteins were isolated by reverse phase HPLC. The protein content was determined using the Bio-Rad protein determination reagent (Bradford method). The fluorescamine assay was done in triplicate.

### 4. Mass Spectrometry

Samples of lysozyme (0.756 mM; 4.54 mM of Lys residues) and D-ribose (50 mM) were incubated in the presence and absence of the selected anti-glycation agents at 37° C. for 5 days and subjected to electrospray mass spectrometry (ESI-MS) in order to characterize the protein intermediates as well as carboxymethylated lysozyme. Prior to MS measurements, the proteins were isolated by reversed phase-HPLC and were semi-dried by freeze drying.

### 5. SDS Polyacrylamide Gel Electrophoresis (SDS-PAGE)

Protein-protein cross-links were characterized by SDS-PAGE. The incubation mixtures used for determination of the amino groups were further incubated for 4 weeks. An aliquot of the solution was applied to Pharmacia SDS FAST gel and the proteins were stained with Coomassie blue.

Although various particular embodiments of the present invention have been described hereinbefore for the purpose of illustration, it would be apparent to those skilled in the art that numerous variations may be made thereto without departing from the spirit and scope of the invention, as defined in the appended claims.

What is claimed:

1. A method comprising administering to a subject an anti-glycation effective amount of one or more compounds of formula (I) 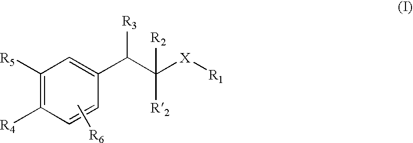
wherein:
X represents NR₇, wherein R₇ represents hydrogen atom or an acyl group derived from a linear or branched aliphatic acid or an aromatic acid,
R₁ represents hydrogen atom, NH₂, or a linear or branched C₁₋₅ alkyl which may be substituted with an aromatic group,
R₂ represents hydrogen atom, a linear or branched C₁₋₅ alkyl, or COOH group,
R′₂ represents hydrogen atom or a linear or branched C₁₋₅ alkyl group,
R₃ represents hydrogen atom, ═O, OR₈, SR₈, or NR₈R₉, wherein R₈ and R₉ represent hydrogen atom, a linear or branched C₁₋₅ alkyl, or an acyl group derived from a linear or branched aliphatic acid or an aromatic acid, provided that R₈ and R₉ are not both an acyl group,
R₄ and R₅ represent OR₁₀, or SR₁₀, wherein R₁₀ represents hydrogen atom or an acyl group derived from a linear or branched aliphatic acid or an aromatic acid,
R₆ represents hydrogen atom, OR₁₀, or SR₁₀, wherein R₁₀ represents hydrogen atom or an acyl group derived from a linear or branched aliphatic acid or an aromatic acid,
their physiologically tolerated salts, prodrugs, physiologically functional derivatives, and mixtures thereof, for the prevention or treatment of glycation in the subject for the prevention or treatment of age-, diabetes- or smoking-related complications.

2. The method according to claim 1, wherein X is NH.

3. The method according to claim 2, wherein R₁ is H, —CH₃, or —CH(CH₃)₂.

4. The method according to claim 3, wherein R₂ is H.

5. The method according to claim 4, wherein R₁₂ is H.

6. The method according to claim 5, wherein R₃ is OH.

7. The method according to claim 6, wherein the compound has D-configuration.

8. The method according to claim 7, wherein R₆ is H and R₄ and R₅ are both OH.

9. The method according to claim 8, wherein the OH groups at positions 3 and 4 of the aromatic ring are pivaloylated (trimethylacetylated).

10. The method according to claim 1, wherein the compound is selected from the group consisting of α-(1-methyl-3-phenyl-propylamino)-3,4-dihydroxyacetophenone, 3,4-dihydroxy-1-[α-(1-methyl-3-phenyl-propylamino)-β-hydroxyethyl]benzene, 3,4-dihydroxy-1-[α-isopropylamino-β-methoxy)ethyl]benzene, 3,4-dihydroxy-1-[(α-amino-β-methoxy)ethyl]benzene, adrenalone, L-DOPA, dopamine, L-epinephrine, isoetharine, D-isoproterenol, L-isoproterenol, L-α-methyl-DOPA, S(−)-carbidopa, D-norepinephrine, L-norepinephrine, 6-hydroxydopamine and corbadrine.

11. The method according to claim 1, wherein the compound is a prodrug or a physiologically functional derivative.

12. The method according to claim 11, wherein the prodrug comprises at least one acyl group derived from a linear or branched aliphatic acid or an aromatic acid.

13. The method according to claim 12, wherein the acyl group acylates at least one of X, R₃, R₄, R₅, or R₆.

14. The method according to claim 13, wherein the acyl group is pivaloyl (trimethylacetyl).

15. The method according to claim 14, wherein X is NH, R₁ is methyl, R₃ is hydroxy, R₂, R′₂ and R₆ are hydrogen, R₄ and R₅ are pivaloylated hydroxy groups, and wherein the compound has D-configuration.

16. The method according to claim 14, wherein X is NH, R₃ is hydroxy, R₁, R₂, R₁₂ and R₆ are hydrogen, R₄ and R₅ are pivaloylated hydroxy groups, and wherein the compound has D-configuration.

17. The method according to claim 14, wherein X is NH, R₁ is isopropyl, R₃ is hydroxy, R₂, R′₂ and R₆ are hydrogen, R₄ and R₅ are pivaloylated hydroxy groups, and wherein the compound has D-configuration.

18. The method according to claim 1 wherein said compound is D-norepinephrine dipivalate.

19. The method according to claim 1 wherein said compound is D-isoproterenol dipivalate.
